(19) United States
(12) Patent Application Publication
(10) Pub. No.: US 2006/0051310 A1
(43) Pub. Date: Mar. 9, 2006

(54) Water-in-oil emulsion comprising a non-volatile non-silicone oil, a cationic surfactant, a polyolefin with polar portion(s), and an alkylmonoglycoside or alkylpolyglycoside

(75) Inventors: Geraldine Fack, Levallois-Perret (FR); Chrystal Pourille-Grethen, Clichy (FR)

Correspondence Address: OBLON, SPIVAK, MCCLELLAND, MAIER & NEUSTADT, P.C., 1940 DUKE STREET, ALEXANDRIA, VA, 22314, US

(73) Assignee: L'OREAL, Paris (FR)

(21) Appl. No.: 11/191,136

(22) Filed: Jul. 28, 2005

Related U.S. Application Data
(60) Provisional application No. 60/609,823, filed on Sep. 15, 2004.

(30) Foreign Application Priority Data
Aug. 2, 2004 (FR) 04/08539

(51) Int. Cl. A61K 8/73; A61K 8/97

(52) U.S. Cl. 424/70.13; 424/74

(57) ABSTRACT

The present invention concerns a cosmetic hair treatment composition, of water-in-oil emulsion type, containing at least one non-volatile non-silicone oil, at least one cationic surfactant, at least one polyolefin with polar portion(s), and at least one of an alkylmonoglycoside and alkylpolyglycoside, and to a cosmetic hair treatment process.

### REFERENCE TO PRIOR APPLICATIONS

This application claims priority to U.S. provisional application 60/609,823 filed Sep. 15, 2004, and to French patent application 04 08539 filed Aug. 2, 2004, both incorporated herein by reference.

### FIELD OF THE INVENTION

The present invention concerns a cosmetic hair treatment composition, of water-in-oil emulsion type, comprising at least one non-volatile non-silicone oil, at least one cationic surfactant, at least one polyolefin with polar portion(s), and at least one of an alkylmonoglycoside and alkylpolyglycoside, and to a cosmetic hair treatment process.

### BACKGROUND OF THE INVENTION

Water-in-oil emulsions, especially those free of silicone compounds, are commonly used in cosmetics and in particular for skincare since they allow a lipid film to be formed on the surface of the skin, thus protecting it against external attack and preventing transepidermal water loss.

However, these emulsions are used very little in the haircare field, and in particular in the field of conditioning of the hair. The reason for this is that they have two major drawbacks, namely that of not being able to be rinsed out easily and completely, leading to an unaesthetic greasy residue, and that of not allowing good conditioning of the hair to be obtained. Specifically, dull, sticky and dirty hair is generally obtained, and softening of the hair fibre is observed. The hair is also difficult to disentangle.

### SUMMARY OF THE INVENTION

The inventors have discovered, surprisingly, that the introduction of a particular combination of surfactants and of polyolefin containing polar portion(s) into a water-in-oil emulsion makes it possible not only to improve its rinseability and to obtain good cosmetic properties, but also to obtain a stable water-in-oil emulsion.

### DETAILED DESCRIPTION OF THE PREFERRED EMBODIMENTS

This particular combination comprises at least one cationic surfactant, at least one polyolefin with polar portion(s), and at least one of a (C₁₂₋₃₀ alkyl)monoglycoside and a (C₁₂₋₃₀ alkyl)polyglycoside, or a mixture thereof.

One subject of the present invention is thus a hair treatment composition, of water-in-oil emulsion type, comprising, preferably in a cosmetically acceptable medium, at least one non-volatile non-silicone oil, at least one cationic surfactant, at least one polyolefin with polar portion(s), and at least one (C₁₂₋₃₀ alkyl)monoglycoside or (C₁₂₋₃₀ alkyl)polyglycoside, or mixture thereof.

Another subject of the invention is a cosmetic hair treatment process using a composition according to the invention as described herein.

A subject of the invention is also the use of the composition according to the invention for conditioning the hair, and especially as a hair conditioner.

Other subjects, characteristics, aspects and advantages of the invention will emerge even more clearly on reading the description and the example that follows.

According to the invention, the cosmetic hair treatment composition, of water-in-oil emulsion type, comprises, preferably in a cosmetically acceptable medium, at least one non-volatile non-silicone oil, at least one cationic surfactant, at least one polyolefin with polar portion(s), and at least one alkylmonoglycoside or alkylpolyglycoside.

The phrase “at least one alkylmonoglycoside or alkylpolyglycoside” includes more than one alkylmonoglycoside, more than one alkylpolyglycoside, and mixtures of alkylmonoglycoside(s) and alkylpolyglycoside(s).

The term “cosmetically acceptable medium” means a medium that is compatible with the hair.

The term “oil” means any non-aqueous medium that is liquid at room temperature (25° C.±3° C.) and atmospheric pressure, with a water solubility at 25° C. of less than 0.5%.

The term “non-volatile oil” means an oil with a vapour pressure at room temperature (25° C.±3° C.) of less than 2.66 Pa (0.02 mmHg).

The non-volatile non-silicone oils that may be used in the present invention include plant oils, animal oils, mineral oils, synthetic oils, fatty acid esters, and mixtures thereof.

Plant oils useful herein include sweet almond oil, avocado oil, castor oil, olive oil, liquid jojoba wax, sunflower oil, wheatgerm oil, sesame seed oil, groundnut oil, grapeseed oil, soybean oil, rapeseed oil, safflower oil, coconut oil, corn oil, hazelnut oil, palm oil, apricot kernel oil and calophyllum oil.

An animal oil that may be mentioned is perhydrosqualene.

The composition according to the invention may also comprise one or more mineral oils such as a liquid paraffin and liquid petrolatum oil.

The composition according to the invention may also comprise one or more synthetic oils such as squalane, poly(α-olefins), for instance isododecane or isohexadecane, transesterified plant oils and fluorinated oils.

The composition according to the invention may also comprise one or more fatty esters, for instance the compounds of formula RₐCOOR_{b} in which Rₐ represents a linear or branched, hydroxylated or non-hydroxylated, saturated or unsaturated fatty acid residue containing from 4 to 29 carbon atoms and R_{b} represents a linear or branched, saturated or unsaturated hydrocarbon-based chain containing from 3 to 30 carbon atoms, the total number of carbon atoms in the ester being greater than 10. Examples that may especially be mentioned include purcellin oil (stearyl octanoate), isopropyl myristate, isopropyl palmitate, butyl stearate, hexyl laurate, isononyl isononanoate, 2-ethylhexyl palmitate, 2-hexyldecyl laurate, 2-octyldecyl palmitate, 2-octyldodecyl myristate, isostearyl neopentanoate and tridecyl neopentanoate.

The oils that are particularly preferred in the composition according to the invention are especially chosen from avocado oil, isododecane, isopropyl myristate and liquid jojoba wax.

The oil(s) as described above is (are) especially present in the composition according to the invention in an amount ranging from 0.1% to 30% by weight, preferably from 1% to 20% by weight and better still from 5% to 15% by weight relative to the total weight of the composition.

The composition according to the invention may comprise one or more cationic surfactants that are well known per se, such as optionally polyoxyalkylenated primary, secondary or tertiary fatty amine salts, quaternary ammonium salts, and mixtures thereof. Examples of quaternary ammonium salts that may especially be mentioned include:
those corresponding to the general formula (I) below: 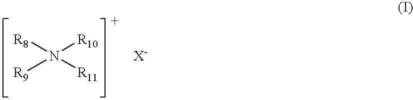
   in which the radicals R₈ to R₁₁, which may be identical or different, represent a linear or branched aliphatic radical containing from 1 to 30 carbon atoms, or an aromatic radical such as aryl or alkylaryl. The aliphatic radicals may comprise hetero atoms such as, especially, oxygen, nitrogen, sulfur and halogens. The aliphatic radicals are chosen, for example, from C₁₋₃₀ alkyl, C₁₋₃₀ alkoxy, C₂-C₆ polyoxyalkylene, C₁₋₃₀ alkylamide, (C₁₂-C₂₂)alkylamido(C₂-C₆)alkyl, (C₁₂-C₂₂)alkylacetate and C₁₋₃₀ hydroxyalkyl radicals; X⁻ is an anion chosen from the group of halides, phosphates, acetates, lactates, (C₂-C₆)alkyl sulfates and alkyl- or alkylaryl-sulfonates.

Among the quaternary ammonium salts of formula (I) that are preferred are, firstly, tetraalkylammonium chlorides, for instance dialkyldimethylammonium or alkyltrimethylammonium chlorides in which the alkyl radical contains from about 12 to 22 carbon atoms, in particular behenyltrimethylammonium chloride, distearyldimethylammonium chloride, cetyltrimethylammonium chloride or benzyldimethylstearylammonium chloride, or, secondly, palmitylamidopropyltrimethylammonium chloride or stearamidopropyldimethyl(myristyl acetate)ammonium chloride sold under the name Ceraphyl® 70 by the company Van Dyk;
quaternary ammonium salts of imidazoline, for instance those of formula (II) below: 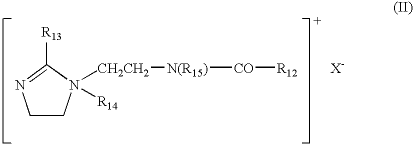
   in which R₁₂ represents an alkenyl or alkyl radical containing from 8 to 30 carbon atoms, for example tallow fatty acid derivatives, R₁₃ represents a hydrogen atom, a C₁-C₄ alkyl radical or an alkenyl or alkyl radical containing from 8 to 30 carbon atoms, R₁₄ represents a C₁-C₄ alkyl radical, R₁₅ represents a hydrogen atom or a C₁-C₄ alkyl radical, and X⁻ is an anion chosen from the group of halides, phosphates, acetates, lactates, alkyl sulfates, alkylsulfonates and alkylarylsulfonates. R₁₂ and R₁₃ preferably represents a mixture of alkenyl or alkyl radicals containing from 12 to 21 carbon atoms, for example tallow fatty acid derivatives, R₁₄ represents a methyl radical and R₁₅ represents a hydrogen atom. Such a product is sold, for example, under the name Rewoquat® W 75 by the company Rewo;
the quaternary diammonium salts of formula (III): 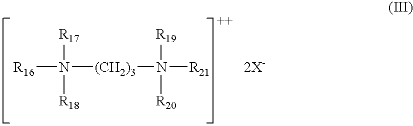
   in which R₁₆ denotes an aliphatic radical containing from about 16 to 30 carbon atoms, R₁₇, R₁₈, R₁₉, R₂₀ and R₂₁, which may be identical or different, are chosen from a hydrogen atom and an alkyl radical containing from 1 to 4 carbon atoms, and X⁻ is an anion chosen from the group of halides, acetates, phosphates, nitrates and methyl sulfates. Such quaternary diammonium salts in particular comprise propanetallowdiammonium dichloride;
quaternary ammonium salts containing at least one ester function, such as those of formula (IV) below: 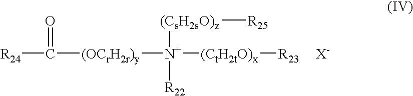
   in which:
R₂₂ is chosen from C₁-C₆ alkyl radicals and C₁-C₆ hydroxyalkyl or dihydroxyalkyl radicals;
R₂₃ is chosen from:
   a radical R₂₆ 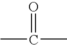
   linear or branched, saturated or unsaturated C₁-C₂₂ hydrocarbon-based radicals R₂₇,
   a hydrogen atom,
R₂₅ is chosen from:
   a radical R₂₈ 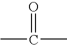
   linear or branched, saturated or unsaturated C₁-C₆ hydrocarbon-based radicals R₂₉,
   a hydrogen atom,
R₂₄, R₂₆ and R₂₈, which may be identical or different, are chosen from linear or branched, saturated or unsaturated C₇-C₂₁ hydrocarbon-based radicals;
r, s and t, which may be identical or different, are integers ranging from 2 to 6;
y is an integer ranging from 1 to 10;
x and z, which may be identical or different, are integers ranging from 0 to 10;
X⁻ is a simple or complex organic or inorganic anion;
   with the proviso that the sum x+y+z is from 1 to 15, that when x is 0, then R₂₃ represents R₂₇ and that when z is 0, then R₂₅ represents R₂₉.

The alkyl radicals R₂₂ may be linear or branched, and more particularly linear.

Preferably, R₂₂ represents a methyl, ethyl, hydroxyethyl or dihydroxypropyl radical, and more particularly a methyl or ethyl radical.

Advantageously, the sum x+y+z is from 1 to 10.

When R₂₃ is a hydrocarbon-based radical R₂₇, it may be long and contain from 12 to 22 carbon atoms, or short and contain from 1 to 3 carbon atoms.

When R₂₅ is a hydrocarbon-based radical R₂₉, it preferably contains 1 to 3 carbon atoms.

Advantageously, R₂₄, R₂₆ and R₂₈, which may be identical or different, are chosen from linear or branched, saturated or unsaturated C₁₁-C₂₁ hydrocarbon-based radicals, and more particularly from linear or branched, saturated or unsaturated C₁-C₂₁ alkyl and alkenyl radicals.

Preferably, x and z, which may be identical or different, are 0 or 1.

Advantageously, y is equal to 1.

Preferably, r, s and t, which may be identical or different, are equal to 2 or 3 and even more particularly equal to 2.

The anion is preferably a halide (chloride, bromide or iodide) or an alkyl sulfate, more particularly methyl sulfate. However, methanesulfonate, phosphate, nitrate, tosylate, an anion derived from an organic acid, such as acetate or lactate, or any other anion that is compatible with the ammonium containing an ester function may be used.

The anion X⁻ is even more particularly chloride or methyl sulfate.

Use is made more particularly in the composition according to the invention of the ammonium salts of formula (IV) in which:
R₂₂ represents a methyl or ethyl radical,
x and y are equal to 1;
z is equal to 0 or 1;
r, s and t are equal to 2;
R₂₃ is chosen from:
   a radical R₂₆ 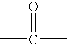
   methyl, ethyl or C₁₄-C₂₂ hydrocarbon-based radicals,
   a hydrogen atom;
R₂₅ is chosen from:
   a radical R₂₈ 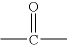
   a hydrogen atom;
R₂₄, R₂₆ and R₂₈, which may be identical or different, are chosen from linear or branched, saturated or unsaturated C₁₃-C₁₇ hydrocarbon-based radicals, and preferably from linear or branched, saturated or unsaturated C₁₃-C₁₇ alkyl and alkenyl radicals.

The hydrocarbon-based radicals are advantageously linear.

Examples of compounds of formula (IV) that may be mentioned include the salts (especially chloride or methyl sulfate) of diacyloxyethyl-dimethylammonium, of diacyloxyethyl-hydroxyethylmethylammonium, of monoacyloxyethyl-dihydroxyethyl-methylammonium, of triacyloxyethyl-methylammonium, of monoacyloxyethyl-hydroxyethyl-dimethylammonium, and mixtures thereof. The acyl radicals preferably contain 14 to 18 carbon atoms and are more preferably derived from a plant oil, for instance palm oil or sunflower oil. When the compound contains several acyl radicals, these radicals may be identical or different.

These products are obtained, for example, by direct esterification of optionally oxyalkylenated triethanolamine, triisopropanolamine, alkyldiethanolamine or alkyldiisopropanolamine onto fatty acids or onto mixtures of fatty acids of plant or animal origin, or by transesterification of the methyl esters thereof. This esterification is followed by a quaternization using an alkylating agent such as an alkyl halide (preferably a methyl or ethyl halide), a dialkyl sulfate (preferably dimethyl or diethyl sulfate), methyl methanesulfonate, methyl para-toluenesulfonate, glycol chlorohydrin or glycerol chlorohydrin.

Such compounds are sold, for example, under the names Dehyquart® by the company Henkel, Stepanquat® by the company Stepan, Noxamium® by the company Ceca, and Rewoquat® WE 18 by the company Rewo-Witco.

The composition according to the invention preferably contains a mixture of quaternary ammonium mono-, di- and triester salts with a weight majority of diester salts.

Examples of mixtures of ammonium salts that may be used include the mixture containing 15% to 30% by weight of acyloxyethyl- dihydroxyethyl-methylammonium methyl sulfate, 45% to 60% of diacyloxyethyl-hydroxyethyl-methylammonium methyl sulfate and 15% to 30% of triacyloxyethyl-methylammonium methyl sulfate, the acyl radicals containing from 14 to 18 carbon atoms and being derived from optionally partially hydrogenated palm oil.

It is also possible to use the ammonium salts containing at least one ester function described in U.S. Pat. No. 4,874,554 and U.S. Pat. No. 4,137,180.

The cationic surfactants that are particularly preferred in the composition of the invention are chosen from quaternary ammonium salts, in particular from behenyltrimethylammonium chloride and cetyltrimethylammonium chloride.

The cosmetic hair treatment composition preferably comprises the cationic surfactant(s) in an amount ranging from 0.1% to 20% by weight, better still from 0.2% to 10% by weight and even more preferably from 0.5% to 8% by weight relative to the total weight of the composition.

The polyolefins with polar portion(s) that may be used in the invention are known in other fields. Thus, they are described, for example, in documents U.S. Pat. No. 5,129,972 and U.S. Pat. No. 4,919,179, as explosive-emulsion stabilizers.

Moreover, these compounds are known as stabilizers for fertiliser compositions (see documents U.S. Pat. No. 5,518,517 and U.S. Pat. No. 5,858,055) for the purpose of obtaining controlled release of the fertilizing substances.

The polyolefins with polar portion(s) used in the composition of the invention comprise a polyolefinic apolar portion and at least one polar portion. They may have a structure of block or comb type.

The polyolefinic apolar portion should contain at least 40 carbon atoms and preferably from 60 to 700 carbon atoms. This apolar portion may be chosen for example from polyolefins such as oligomers, polymers and/or copolymers of ethylene, propylene, 1-butene, isobutene, 1-pentene, 2-methyl-1-butene, 3-methyl-1-butene, 1-hexene, 1-heptene, 1-octene, 1-decene, 1-undecene, 1-dodecene, 1-tridecene, 1-tetradecene, 1-pentadecene, 1-hexadecene, 1-heptadecene and 1-octadecene. These polyolefins are hydrogenated or non-hydrogenated.

Moreover, the polyolefins with polar portion(s) used in the composition of the invention comprise at least one polar portion. This polar portion gives them amphiphilic properties. Thus, these polyolefins with polar portion(s) lower the interface tension (water/oil) by at least 10 mN/m when they are present at a concentration of 0.01% by weight relative to the total weight of the oily phase. For example, the polyolefin with succinic end group sold under the name Lubrizol 2724 by the company Lubrizol, at a concentration of 0.01% by weight relative to the total weight of the oily phase, lowers the interface tension by 15 mN/m at the interface of an aqueous phase consisting of an aqueous 1% MgSO₄ solution, and of an oily phase comprising a mixture of oils (isohexadecane/hydrogenated polyisobutene/volatile silicone in an 8/6/4 ratio).

The polar portion of the polyolefins with polar portion(s) of the invention may be anionic, cationic, nonionic, zwitterionic or amphoteric. It comprises, for example, of polyalkylene glycols or polyalkylene imines, or alternatively of carboxylic or dicarboxylic acids, anhydrides thereof or derivatives thereof, such as esters thereof, amides thereof and salts thereof, and mixtures thereof. The polyolefins with a carboxylic acid polar portion may be derived, for example, from the reaction between a polyolefin and at least one carboxylic acid or anhydride that is optionally partially or totally salified, chosen from the group comprising maleic acid, maleic anhydride, fumaric acid, itaconic acid, citraconic acid, mesaconic acid, aconitic acid and succinic acid or anhydride, ester or amide derivatives thereof, and mixtures thereof.

Preferably, the polar portion consists of succinic acid or anhydrsalts, alkaline-earth metal salts or organic salts of succinic acid or anhydride, or partial salts of monoesters or monoamides of succinic acid or anhydride, or alternatively of a polyoxyethylene.

The polyolefins with polyoxyethylene polar portion(s) may be chosen, for example, from polyisoprene-polyoxyethylene diblock polymers and poly(ethylene-co-propylene)-polyoxyethylene polymers, and mixtures thereof. These polymers are described in the publication by Allgaier, Poppe, Willner and Richter (Macromolecules, 1997, Vol. 30, pp. 1582-1586).

The polyolefins with a succinic acid or anhydride polar portion may be chosen especially from the polyolefins derived from succinic acid or anhydride described in patents U.S. Pat. No. 4,234,435, U.S. Pat. No. 4,708,753, U.S. Pat. No. 5,129,972, U.S. Pat. No. 4,931,110, GB-A-2 156 799 and U.S. Pat. No. 4,919,179. The polyolefin portion may comprise, for example, of hydrogenated or non-hydrogenated polyisobutylene with a molecular weight ranging from 400 to 5000. In the polyisobutylene containing succinic end group thus obtained, the succinic portion may be optionally modified, i.e. esterified, amidated or in salt form. It may be modified with alcohols, amines, alkanolamines or polyols, or alternatively may be in the form of alkali metal, alkaline-earth metal or ammonium salts or alternatively in the form of organic-base salts, for instance diethanolamine and triethanolamine salts. The polyolefins with esterified or amidated succinic end group are products of reaction of (a) a polyolefinic with succinic end group and of (b) an amine or an alcohol, to form an amide or an ester. The term “amine” used herein includes all types of amine, including alkanolamines. The amines may be, for example, primary, secondary or tertiary monoamines, these amines possibly being aliphatic, cycloaliphatic, aromatic or heterocyclic, and saturated or unsaturated. Moreover, the alcohols may be monoalcohols or polyalcohols. The monoalcohols comprise primary, secondary or tertiary aliphatic alcohols, and phenols. The polyalcohols may be chosen, for example, from aliphatic, cycloaliphatic, aromatic and heterocyclic polyalcohols. The polyolefins with modified (esterified or amidated) succinic end group and the process for preparing them are described in particular in document U.S. Pat. No. 4,708,753.

Polyolefins with succinic end group that may especially be mentioned include polyisobutylenes with esterified succinic end group and salts thereof, especially the diethanolamine salts, such as the products sold under the names Lubrizol 2724, Lubrizol 2722 and Lubrizol 5603 by the company Lubrizol.

Another example of a polyolefin with polar portion(s) that may be used in the invention is the product of reaction of maleic anhydride with polyisobutylene, such as the product sold under the name Glissopal SA by the company BASF.

The polyolefin with polar portion(s) that is particularly preferred is a product of reaction of polyisobutenylsuccinic anhydride with diethylethanolamine. This product is sold, for example, under the name Lubrizol 5603 by the company Lubrizol, and may be represented by the following formula: 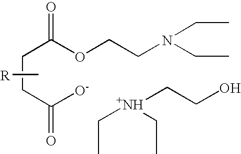
in which R represents a polyisobutenyl group with a weight-average molecular mass of 1000.

The polyolefins(s) with polar portion(s) as defined above is (are) preferably contained in an amount ranging from 0.01% to 10% by weight, preferably from 0.1% to 5% by weight and better still from 0.2% to 3% by weight relative to the total weight of the composition.

The alkylmonoglycosides or alkylpolyglycosides that are particularly preferred in the invention are those in which the alkyl group contains from 16 to 24 carbon atoms.

A particularly preferred example that may especially be mentioned is arachidylglycoside.

The (C₁₂₋₃₀ alkyl)monoglycoside(s) or (C₁₂₋₃₀ alkyl)polyglycosides is (are) preferably contained in an amount ranging from 0.01% to 10% by weight, preferably from 0.02% to 5% by weight and better still from 0.05% to 1% by weight relative to the total weight of the composition.

Preferably, the weight ratio of oil(s)/polyolefins(s) with polar portion(s) in the compositions of the invention is between 3 and 0.100, better still between 10 and 75 and even more preferably between 15 and 40.

The cosmetically acceptable aqueous medium may comprise for example water or a mixture of water and of a cosmetically acceptable solvent chosen from C₁-C₄ lower alcohols, such as ethanol, isopropanol, tert-butanol or n-butanol; polyols, for instance propylene glycol; polyol ethers; C₅-C₁₀ alkanes; C₃₋₄ ketones, for instance acetone and methyl ethyl ketone; C₁-C₄ alkyl acetates, for instance methyl acetate, ethyl acetate and butyl acetate; dimethoxyethane and diethoxyethane; and mixtures thereof.

The compositions according to the invention may also comprise at least one C₁₄₋₃₀ fatty alcohol, and preferably at least one alcohol from among myristyl alcohol, cetyl alcohol, stearyl alcohol, arachidyl alcohol, behenyl alcohol and erucyl alcohol. They are generally present in an amount of less than 10% by weight, preferably ranging from 0.01% to 5% by weight and better still from 0.05% to 1.5% by weight relative to the total weight of the composition.

The compositions according to the invention may also comprise at least one silicone oil that is well known in the art, in an amount of less than 10% by weight, preferably ranging from 0.01% to 8% and even more preferably from 0.1% to 5% by weight relative to the total weight of the composition.

Examples of silicone oils that may especially be mentioned include linear or cyclic polydimethylsiloxanes.

The compositions according to the invention may also contain at least one additive such as a cationic, anionic, nonionic or amphoteric polymer; a natural or synthetic anionic, amphoteric, zwitterionic, nonionic or cationic, associative or non-associative polymeric thickener; a non-polymeric thickener, for instance an electrolyte or a sugar; a nacreous agent; an opacifier; a sunscreen; a fragrance; a dye; an organic or mineral particle; a preserving agent; a pH stabilizer.

A person skilled in the art will take care to select the optional additives and the amount thereof such that they do not harm the properties of the compositions of the present invention.

These additives are present in the composition according to the invention in an amount ranging for example from 0 to 50% by weight relative to the total weight of the composition.

The compositions according to the invention may be in the form of fluid or thickened liquids, gels, creams, or simple or multiple emulsions.

The compositions may be used, for example, in shampoos, dyeing, bleaching or permanent-waving products, styling products, rinse-out care products, deep-care masks, shower gels, lotions or creams for treating the scalp, or alternatively deposited onto wipes.

The present invention also relates to a cosmetic hair treatment process that comprises applying an amount of a composition as described above to the hair, and optionally rinsing it out after an optional leave-in time.

According to one preferred embodiment of the invention, the composition may be used for conditioning the hair and more particularly as a hair conditioner.

The example that follows is given as an illustration of the present invention.

### EXAMPLE

The water-in-oil emulsion was prepared by mixing together the ingredients indicated in the table below in the indicated weight percentages relative to the total weight of the emulsion.

| | | Ex. 1 |
|---|---|---|
| C₁₁₋₁₂ isoparaffin | | 3.55 |
| Avocado oil | | 1.2 |
| Polyolefin with a polar portion⁽¹⁾ | | 0.25 |
| Arachidylglucoside (at 15% AM)⁽²⁾ | | 0.2 AM |
| Behenyltrimethylammonium chloride (at 80% AM)⁽³⁾ | | 2 AM |
| Water | qs | 100 |

AM: Active material
⁽¹⁾sold under the trade name Lubrizol TM 5603 by Lubrizol
⁽²⁾sold under the trade name Montanov 202 by Seppic
⁽³⁾sold under the trade name Genamin KDMP by Clariant

The emulsion was applied to sensitized hair.

Good rinseability and good conditioning of the fibres are then observed.

The above written description of the invention provides a manner and process of making and using it such that any person skilled in this art is enabled to make and use the same, this enablement being provided in particular for the subject matter of the appended claims, which make up a part of the original description and including a composition in the form of a water-in-oil emulsion, comprising, in a cosmetically acceptable medium, at least one non-volatile non-silicone oil, at least one cationic surfactant, at least one polyolefin with polar portion(s), and at least one material selected from (C₁₂₋₃₀ alkyl)monoglycosides and (C₁₂₋₃₀ alkyl)polyglycosides.

As used above, the phrases “selected from the group consisting of,” “chosen from,” “selected from,” and the like include mixtures of the specified materials.

All references, patents, applications, tests, standards, documents, publications, brochures, texts, articles, etc. mentioned herein are incorporated herein by reference. Where a numerical limit or range is stated, the endpoints are included. Also, all values and subranges within a numerical limit or range are specifically included as if explicitly written out. Terms such as “contain(s)” and the like as used herein are open terms meaning ‘including at least’ unless otherwise specifically noted.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

1. A composition in the form of a water-in-oil emulsion, comprising at least one non-volatile non-silicone oil, at least one cationic surfactant, at least one polyolefin with polar portion(s), and at least one material selected from (C₁₂₋₃₀ alkyl)monoglycosides and (C₁₂₋₃₀ alkyl)polyglycosides.

2. A composition according to claim 1, wherein the non-volatile non-silicone oil is a plant oil, an animal oil, a mineral oil, a synthetic oil, a fatty acid ester, or a mixture thereof.

3. A composition according to claim 2, comprising at least one plant oil chosen from sweet almond oil, avocado oil, castor oil, olive oil, liquid jojoba wax, sunflower oil, wheatgerm oil, sesame seed oil, groundnut oil, grapeseed oil, soybean oil, rapeseed oil, safflower oil, coconut oil, corn oil, hazelnut oil, palm oil, apricot kernel oil and calophyllum oil.

4. A composition according to claim 2, comprising perhydrosqualene.

5. A composition according to claim 2, comprising at least one mineral oil chosen from liquid paraffin and petrolatum oil.

6. A composition according to claim 2, comprising at least one synthetic oil is chosen from squalane, poly(α-olefins), transesterified plant oils and fluorinated oils.

7. A composition according to claim 2, comprising at least one fatty ester chosen from purcellin oil, isopropyl myristate, isopropyl palmitate, butyl stearate, hexyl laurate, isononyl isononanoate, 2-ethylhexyl palmitate, 2-hexyldecyl laurate, 2-octyldecyl palmitate, 2-octyldodecyl myristate, isostearyl neopentanoate and tridecyl neopentanoate.

8. A composition according to claim 1, wherein the non-volatile non-silicone oil is chosen from avocado oil, isododecane, isopropyl myristate, liquid jojoba wax and mixtures thereof.

9. A composition according to claim 1, wherein it comprises the oil(s) in an amount ranging from 0.1% to 30% by weight relative to the total weight of the composition.

10. A composition according to claim 1, wherein the cationic surfactants are chosen from optionally polyoxyalkylenated primary, secondary or tertiary fatty amine salts, quaternary ammonium salts, and mixtures thereof.

11. A composition according to claim 10, comprising at least one quaternary ammonium salt are chosen from:
those corresponding to the general formula (I) below: 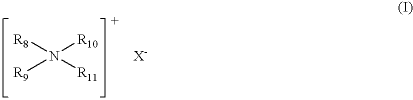
in which the radicals R₈ to R₁₁, which may be identical or different, represent a linear or branched aliphatic radical containing from 1 to 30 carbon atoms, or an aromatic radical; X⁻ is an anion chosen from the group of halides, phosphates, acetates, lactates, (C₂-C₆)alkyl sulfates and alkyl- or alkylaryl-sulfonates;
quaternary ammonium salts of imidazoline;
the quaternary diammonium salts of formula (III): 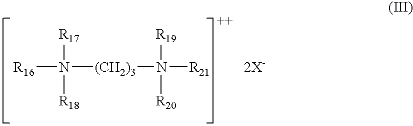
in which R₁₆ represents an aliphatic radical containing from about 16 to 30 carbon atoms, R₁₇, R₁₈, R₁₉, R₂₀ and R₂], which may be identical or different, are chosen from a hydrogen atom and an alkyl radical containing from 1 to 4 carbon atoms, and X⁻ is an anion chosen from the group of halides, acetates, phosphates, nitrates and methyl sulfates;
quaternary ammonium salts containing at least one ester function.

12. A composition according to claim 1, comprising at least one cationic surfactant chosen from behenyltrimethylammonium chloride and cetyltrimethylammonium chloride.

13. A composition according to claim 1, comprising the cationic surfactant(s) in an amount ranging from 0.1% to 20% by weight relative to the total weight of the composition.

14. A composition according to claim 1, wherein the polyolefin with polar portion(s) comprises a polyolefinic apolar portion containing at least 40 carbon atoms.

15. A composition according to claim 14, wherein the polyolefinic apolar portion is chosen from oligomers, polymers and/or copolymers of ethylene, propylene, 1-butene, isobutene, 1-pentene, 2-methyl-1-butene, 3-methyl-1-butene, 1-hexene, 1-heptene, 1-octene, 1-decene, 1-undecene, 1-dodecene, 1-tridecene, 1-tetradecene, 1-pentadecene, 1-hexadecene, 1-heptadecene and 1-octadecene.

16. A composition according to claim 1, wherein the polar portion is anionic, cationic, nonionic, zwitterionic or amphoteric.

17. A composition according to claim 1, wherein the polar portion consists of polyalkylene glycols, polyalkylene imines, carboxylic or dicarboxylic acids, anhydrides or derivatives thereof such as esters, amides and salts, and mixtures thereof.

18. A composition according to claim 1, wherein the polar portion is chosen from the group comprising polyoxyethylene, succinic acid or anhydride, succinic acid or anhydride esters or amides, alkali metal salts, alkaline-earth metal salts or organic salts of succinic acid or anhydride, or partial salts of succinic acid or anhydride monoesters or monoamides.

19. A composition according to claim 1, wherein the polyolefin with polar portion(s) is a polyisobutylene with optionally modified succinic end group.

20. A composition according to claim 1, wherein the polyolefin with polar portion(s) is the product of reaction of maleic anhydride with polyisobutylene.

21. A composition according to claim 1, wherein it comprises the polyolefin(s) with polar portion(s) in an amount ranging from 0.01% to 10% by weight relative to the total weight of the composition.

22. A composition according to claim 1, wherein the (C₁₂₋₃₀ alkyl)monoglycoside and (C₁₂₋₃₀ alkyl)polyglycoside are such that the alkyl group contains from 16 to 24 carbon atoms.

23. A composition according to claim 1, wherein it comprises the (C₁₂₋₃₀ alkyl)monoglycoside(s) and (C₁₂₋₃₀ alkyl)polyglycoside(s) in an amount ranging from 0.01% to 10% by weight relative to the total weight of the composition.

24. A composition according to claim 1, wherein the weight ratio of oil(s)/polyolefin(s) with polar portion(s) is 3-100.

25. A composition according to claim 1, wherein it further comprises at least one C₁₄₋₃₀ fatty alcohol.

26. A composition according to claim 25, wherein the C₁₄₋₃₀ fatty alcohol is chosen from myristyl alcohol, cetyl alcohol, stearyl alcohol, arachidyl alcohol, behenyl alcohol, erucyl alcohol and mixtures thereof.

27. A composition according to claim 25, wherein it comprises the fatty alcohol(s) in an amount of less than 10% by weight relative to the total weight of the composition.

28. A composition according to claim 1, further comprising at least one cosmetically acceptable solvent other than water.

29. A composition according to claim 28, wherein the cosmetically acceptable solvent is chosen from C₁-C₄ lower alcohols, polyols, polyol ethers, C₅-C₁₀ alkanes, C₃₋₄ ketones, C₁-C₄ alkyl acetates, dimethoxyethane, diethoxyethane and mixtures thereof.

30. A composition according to claim 1, wherein it further comprises at least one silicone oil in an amount of less than 10% by weight relative to the total weight of the composition.

31. A composition according to claim 1, wherein it further comprises at least one additive chosen from cationic, anionic, nonionic or amphoteric polymers; natural or synthetic, anionic, amphoteric, zwitterionic, nonionic or cationic, associative or non-associative polymeric thickeners; non-polymeric thickeners; nacreous agents; opacifiers; sunscreens; fragrances; dyes; organic or mineral particles; preserving agents; pH stabilizers, and mixtures thereof.

32. A process, comprising applying to hair the composition of claim 1.

33. The process of claim 32, wherein said composition is a hair conditioner.
